(19) United States
(12) Patent Application Publication
(10) Pub. No.: US 2002/0193694 A1
(43) Pub. Date: Dec. 19, 2002

(54) Apparatus for characterizing the condition of a myocardium

(75) Inventors: Oleg Anosov, Erlangen (DE); Serguej Berdychev, Erlangen (DE); Bernhard Hensel, Erlangen (DE); Ildar Khassanov, Erlangen (DE); Max Schaldach, Berlin (DE)

Correspondence Address: HAHN LOESER & PARKS, LLP, TWIN OAKS ESTATE, 1225 W. MARKET STREET, AKRON, OH, 44313, US

(73) Assignee: Biotronik Mess- und Therapiegeraete GmbH & Co

(21) Appl. No.: 10/101,765

(22) Filed: Mar. 19, 2002

(30) Foreign Application Priority Data
Mar. 21, 2001 (DE) 101 14 724.4

(51) Int. Cl. A61B 5/04

(52) U.S. Cl. 600/509; 600/508

(57) ABSTRACT

An apparatus for characterizing a condition of a myocardium comprising an excitation wave detector which detects an electrical excitation wave propagated through the myocardium at a first (r₁) and second point (r₂) of the myocardium as a first signal (S₁(t)) and a second signal (S₂(t)), and an analysis means which is connected to the excitation wave detector and analyzes the first signal (S₁(t)) and the second signal (S₂(t)), wherein the analysis means detects a difference between a signal shape of the first signal (S₁(t)) and the second signal (S₂(t)).

The present invention concerns an apparatus for characterizing a condition of a myocardium comprising an excitation wave detector which is adapted to detect an electrical excitation wave propagating through the myocardium at a first and second point of the myocardium as a first and second signal, and an analysis means which is connected to the excitation wave detector and adapted to analyze the first and second signals. The invention further concerns a method of operating an analysis apparatus comprising the step of detecting an electrical excitation wave at a first and second point of the myocardium as a first and second signal.

### BACKGROUND OF THE ART

The characterization of the condition of the myocardium is an important matter of concern for the myocardium or heart muscle provides for periodic contraction of the chamber of the heart and thus guarantees the necessary circulation of blood through the body. Faults or defects in the myocardium result in the pumping capacity of the heart being adversely affected and ultimately such a defect can result in the heart stopping.

Regular beating of the heart is to be attributed to the inherent rhythmicity of the heart musculature. No controlling nerves are to be found in the heart and an external regulating mechanism is not necessary to cause the heart muscle to contract rhythmically. The rhythm of the heart beat originates from the heart itself. It can be demonstrated for example under laboratory conditions that fragments of the heart musculature continue to contract rhythmically. That intensic capability however is not adequate to permit efficient functioning of the heart. For that purpose, it is necessary to co-ordinate the muscle contraction in the heart. That is effected by means of a conduction system in the heart, which primarily comprises two nodes comprising specialist tissue, from which pulses issue, and a conduction system for the transmission of those pulses, the ends thereof extending to the inside surface of the ventricle. The rate at which the heart contracts and the synchronization of atrial and ventricular contraction which is necessary for an effective blood pumping effect depends on the electrical properties of the myocardium cells and the conduction of electrical pulses from one region of the heart into another. Therefore, characterizing that excitation conduction affords information about the condition of the myocardium.

Characterization of the conduction property of the myocardium is conventionally effected by the electrical potential of the myocardium being measured at two points thereof. An excitation wave which is propagated between those two points produces the signals which are picked up at the two points. Accordingly those signals reproduce the arrival of the excitation wave at the first and second points. Finally, the two signals which are recorded are compared to each other by means of an analysis device. Thus, the speed of propagation of the excitation wave between the two measurement points in the myocardium can be ascertained from the spacing in respect of time between the occurrence of the signals and the distance of the points in the myocardium where the signals were picked up. A disturbance in or modification of the conduction properties of the myocardium is expressed for example by virtue of the fact that the spacing in respect of time between the recorded signal changes, or a second signal cannot be measured after a first signal has been recorded. The latter indicates that excitation conduction between the two points is totally interrupted.

The above-mentioned conventional apparatuses for characterizing the condition of a myocardium however permit only few conclusions to be drawn about the condition of the myocardium for they only take account of the speed of propagation and the weakening of the excitation wave as it is propagated. It is to be assumed however that the recorded signals can supply a large number of items of information which can provide data about the condition of the myocardium.

### SUMMARY OF THE INVENTION

Therefore the object of the present invention is to provide an apparatus and a method of characterizing a condition of a myocardium, which make it possible to more accurately characterize the influence of the myocardium on the propagation of excitation waves.

That object is attained by an apparatus for characterizing a condition of a myocardium comprising an excitation wave detector which is adapted to detect an electrical excitation wave which is propagated through the myocardium at a first (r₁) and second point (r₂) of the myocardium as a first signal (S₁(t)) and a second signal (S₂(t)), and an analysis means which is connected to the excitation wave detector and adapted to analyze the first signal (S₁(t)) and the second signal (S₂(t)), wherein the analysis means is adapted to detect a difference between the signal shapes of the first signal (S₁(t)) and the second signal (S₂(t)).

The invention is therefore directed to detecting and comparing the signal morphology of an excitation wave which is detected at two different locations and which therefore furnishes two signals. It is advantageous in regard to the apparatus according to the invention in particular that it makes it possible to characterize the influence of the condition of the myocardium on the form of a propagating excitation wave. It is to be assumed that specific properties of the myocardium have an influence on the variation in form so that the detected difference between the signal shapes permits characterization of the condition of the myocardium. Upon propagation of the excitation wave it “disperses”, that is to say the situation involves dispersion of the signal which is being propagated, which can be ascertained by comparison of the signal morphologies or signal shape recorded at two different locations, and can be further evaluated.

The analysis means preferably includes at least one parameter unit which is adapted to characterize the signal shapes of the first (S₁(t)) and the second signal (S₂(t)) on the basis of at least one parameter. The difference between the values of the parameter respectively ascertained for the first and second signal is then a suitable measurement in respect of the characteristic variation in the form of the detected excitation wave between the points r₁ and r₂. Such a parameter can represent for example the half-value width or the maximum gradient of the signals detected at the points r₁ and r₂. It is self-evidently also possible to characterize the signal shape by means of a plurality of different parameters, and to compare them to each other.

For analysis of the respective signal shape, preferably both the first and also the second signal are represented by a superimposition of a set of functions. Therefore, the analysis means is preferably adapted to represent the first signal (S₁(t)) and the second signal (S₂(t)) by superimposition of a set of functions {f(wt)} with wεR, wherein

$${S}_{1} ( t ) = {\int }_{- ∞}^{∞} {C}_{1} ( w ) f ( wt ) ⅆ w and {S}_{2} ( t ) = {\int }_{- ∞}^{∞} {C}_{2} ( w ) f ( wt ) ⅆ w$$

The selected family of functions {f(wt)} must be suitable for representing the recorded signals. It can be mathematically demonstrated and is known that such representations exist. The capability of representation of the recorded signals is based on the fact that they are square-integratable, that is to say the area under the squared signals is finite. The set of the square-integratable functions forms a vector space. If the family of functions {f(wt)} forms a base of that vector space, then any desired signal can be represented by those functions. Each family of functions which defines the vector space of the square-integratable function is thus suitable for representing the recorded signals. The advantage of such a representation is that a variation in the signal shape is expressed in a variation in the functions Cⱼ(w)f(wt) (ᵢ=1,2). The influence of the myocardium on propagation of the excitation wave can thus be interpreted as influencing or varying each individual function of the family of functions.

The analysis means preferably has a Fourier analysis unit. That unit is adapted to implement Fourier analysis, that is to say the exponential functions exp(iwt) are used for the functions f(wt). The coefficients Cⱼ(w) (j=1,2) are then calculated in accordance with

$${C}_{j} ( w ) = {\int }_{- ∞}^{∞} {S}_{j} ( t ) exp ( - ⅈ wt ) ⅆ t .$$

The above-indicated integrals are also identified as Fourier transforms. The coefficients are therefore generally complex-valued. The signals Sⱼ(t) (j=1,2) can thus be represented by means of

$${S}_{j} ( t ) = {\int }_{- ∞}^{∞} {C}_{j} ( w ) exp ( ⅈ wt ) ⅆ w ,$$

which is equivalent to S

$${S}_{j} ( t ) = {\int }_{- ∞}^{∞} {D}_{j} ( w ) cos ( wt + {e}_{j} ( w ) ) ⅆ w .$$

Dⱼ(w)cos(wt+ej(w))dw. Dj(w) represents the amplitude spectrum and eⱼ(w) represents the phase spectrum of the recorded signal. Thus, differences in the signal shape of the first (j=1) and the second (j=2) signals can be interpreted as attenuation and phase shifting of a Fourier component Cⱼ(w)exp(−iwt). The analysis means therefore preferably includes an attenuation analysis unit which is connected to the Fourier analysis unit and is adapted to ascertain attenuation δ(w) of a Fourier component between the points r₁ and r₂. In addition the analysis means preferably has a speed analysis unit which is connected to the Fourier analysis unit and adapted to ascertain a phase speed vₚ(w) of a Fourier component. The phase speed denotes the speed at which a Fourier component is propagated through the myocardium. The speed can be the same for all Fourier components or different for each of the Fourier components. The latter results in a change in the signal shape and is referred to as dispersion. The phase speed can be ascertained by means of the phase change e(w) between the corresponding Fourier component of the first and second signals. The phase shift is greater in proportion to an increasing distance between the measurement parts and smaller in proportion to an increasing phase speed.

The above-described Fourier analysis procedure is only one of many possible methods of representation for the recorded signals. It is also possible for the recorded signals to be represented by means of wavelet analysis. For that purpose the analysis means includes a wavelet analysis unit. The wavelet transform of a signal S(t) is given by

$$C ( a , b ) = {\int }_{- ∞}^{∞} {a}^{- 1 / 2} S ( t ) Ψ ( ( t - b ) / a ) ⅆ t .$$

Unlike the Fourier transform the wavelet transform C(a,b) is a function of two different parameters a and b. The function ψ((t−b)/a) represents a so-called wavelet. In terms of definition is so selected that by means of the transform

$$S ( t ) = {\sum }_{k = - ∞}^{∞} {\int }_{- ∞}^{∞} {a}_{k}^{- 1 / 2} C ( {a}_{k} , b ) Ψ ( \frac{t - b}{{a}_{k}} ) ⅆ b ( wherein {a}_{k} = {2}^{k} )$$

the original signal S(t) is obtained again. The wavelet components sₐ(t) can be defined as follows:

$${s}_{a} ( t ) = {\int }_{- ∞}^{∞} {a}^{- 1 / 2} C ( a , b ) Ψ ( \frac{t - b}{a} ) ⅆ b .$$

The original signal S(t) can be represented as a superimposition of those wavelet components sₐ(t). That representation can be ascertained in each case for the signals recorded at the points r₁ and r₂, wherein the wavelet components for the different signals at the points r₁ and r₂ differ from each other.

Preferably the apparatus according to the invention includes an attenuation analysis unit which is connected to the wavelet analysis unit and adapted to ascertain an attenuation

$$δ ( a ) = {\int }_{- ∞}^{∞} {s}_{a , 2}^{2} ( t ) ⅆ t / {\int }_{- ∞}^{∞} {s}_{a , 1}^{2} ( t ) ⅆ t$$

of the wavelet components s_{a,1}(t) and S_{a,2}(t) between the points (r₁ and r₂). (s_{a,1}(t) and S_{a,2}(t) denote the function sₐ(t) for the points r₁ and r₂). The wavelet components of the signals recorded at the points r₁ and r₂ are respectively functions of time t and a parameter a. Those wavelet components which involve the same parameter value a are identified with each other and for same an attenuation effect is ascertained between the points r₁ and r₂.

Furthermore the speed analysis unit is preferably connected to the wavelet analysis unit and adapted to ascertain a phase speed vₚ(a) of the wavelet component sₐ(t). That is effected by ascertaining the quotient from the spacing between the points r₁ and r₂ and the zero-passages t_{a,2} and t_{a,1} of the wavelet components.

In addition the speed analysis unit can be adapted to ascertain a group speed v_{g}(a) of the wavelet component sₐ(t). For that purpose firstly the respective envelopes A_{a,1}(t) and A_{a,2}(t) of the wavelet component sₐ(t) of the signals S₁(t) and S₂(t) are calculated. The envelopes A_{a,1}(t) and A_{a,2}(t) can be calculated on the basis of

$${A}_{a , j} = {[ {s}_{a , j}^{2} ( t ) + {\hat{s}}_{a , j}^{2} ( t ) ]}^{1 / 2} ( j = 1 , 2 ) ,$$

wherein Ŝ_{a,j}²(t) is given by the Hilbert transformation

$${\hat{s}}_{a , j} ( t ) = - {π}^{- 1} {\int }_{- ∞}^{∞} \frac{{s}_{a , j} ( t )}{τ - t} ⅆ τ .$$

The envelopes of the wavelet component sₐ(t) of the signals S₁(t) and S₂(t) each have a maximum at the times τ_{a,2} and τ_{a,1}. The group speed of the wavelet component then arises out of the spacing between the locations r₁ and r₂ of the recorded signals and the spacing in respect of time between the maxima of the envelopes of the wavelet component of the signals S₁(t) and S₂(t).

Finally the analysis means of the apparatus according to the invention preferably has a refractive index analysis unit which ascertains a value analogous to the refractive index n(a)=v_{g}(a)/vₚ(a) in optics. The refractive index analysis unit is connected to the speed analysis unit and adapted to ascertain a quotient from group speed and phase speed of a wavelet component.

By analogy with optics, a complex refractive index of a wavelet component can be ascertained as by n(a)+iδ(a) from the refractive index n(a) and the attenuation δ(a) of a wavelet component.

In addition the excitation wave detector of the apparatus according to the invention includes first and second electrodes for detecting the first signal S₁(t) and S₂(t) which are also suitable for being placed endocardially or epicardially.

Preferably the apparatus includes a signal store which is connected to the excitation wave detector and the analysis means and is adapted to provide for intermediate storage of the first and second signals. Detection and analysis of the recorded signals can thus be implemented separately from each other in respect of time.

Finally the analysis means of the apparatus according to the invention is preferably adapted to represent the first and second signals separately for each cardiac cycle by superimposition of the set of the functions {f(wt)}. The recorded signals thus only reproduce a propagating excitation wave at two points r₁ and r₂.

### BRIEF DESCRIPTION OF THE DRAWINGS

A preferred embodiment of the present invention is described with reference to the accompanying Figures in which:

FIG. 1 shows a block circuit diagram of an embodiment of the apparatus according to the invention for characterizing a condition of a myocardium,

FIG. 2 is a diagrammatic view of the arrangement of the electrodes of the first embodiment for recording electrical signals from the myocardium,

FIG. 3 shows a wavelet component sₐ(t) and the envelope Aₐ(t) thereof,

FIG. 4 shows two wavelet components recorded at the same time at different points of the myocardium and the shift in respect of time of the zero-passages thereof, and

FIG. 5 shows the envelopes of the two wavelet components recorded at the same time at different points of the myocardium and the shift in respect of time of the maxima thereof.

### DETAILED DESCRIPTION OF THE INVENTION

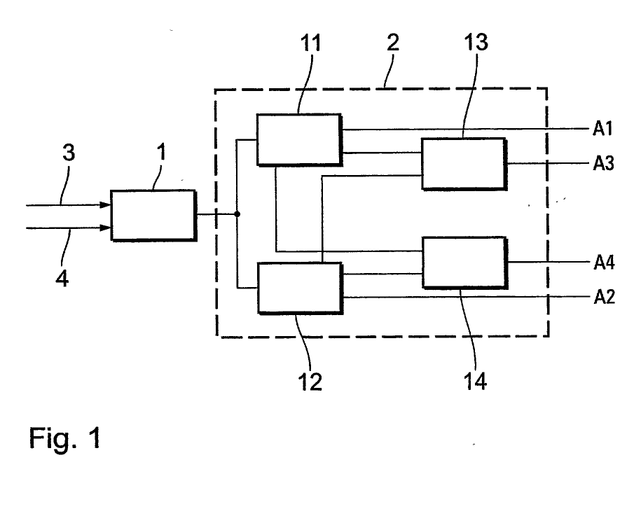

Described hereinafter is the block circuit diagram shown in FIG. 1 of the apparatus according to the invention for characterizing a condition of a myocardium. The apparatus includes an excitation wave detector 1 and an analysis means 2, which are connected together. The excitation wave detector 1 includes two lines 3 and 4 for recording signals from the myocardium. The signals which are recorded at the same time are detected by the excitation wave detector 1 and forwarded to the analysis means 2. The latter has either a Fourier analysis unit 11 or a wavelet analysis unit 12 or both a Fourier analysis unit 11 and also a wavelet analysis unit 12. The recorded signals can be subjected either to Fourier analysis or wavelet analysis or both Fourier analysis and also wavelet analysis. The Fourier analysis unit 11 provides that the recorded signal is developed in accordance with the spectral constituents thereof. The result of that analysis procedure, that is to say the frequency spectrum of the recorded signal, can be outputted by way of an output A1. In contrast the wavelet analysis unit 12 develops the recorded signals in accordance with the wavelet components thereof, which can be outputted by way of the output A2. The Fourier analysis unit and the wavelet analysis unit are respectively connected to a speed analysis unit and an attenuation analysis unit. They are adapted to ascertain the speed of propagation or phase speed or attenuation of the Fourier and wavelet components respectively. The ascertained results of the speed analysis unit 13 and the attenuation analysis unit 14 are respectively outputted by way of the outputs A3 and A4.

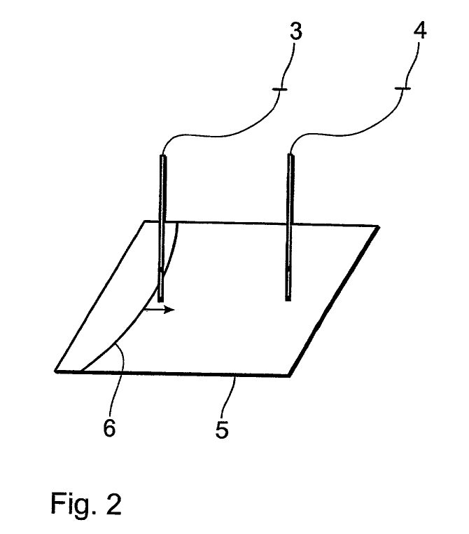

FIG. 2 shows an arrangement of the electrodes for recording the excitation wave signals at two points in accordance with the preferred embodiment. Illustrated here is a portion of the myocardium 5 over which an electrical excitation wave 6 is propagated in the direction indicated by the arrow. Two electrodes 1 and 2 are disposed at a spacing from each other on the myocardium 5. The excitation wave 6 is propagated in the direction of the notional connecting line between the electrodes 1 and 2. The time delay between reception of the excitation wave 6 at the electrode 1 and the electrode 2 thus affords information about the speed of propagation of the excitation wave 6 in the direction of the connecting line between the electrodes 1 and 2.

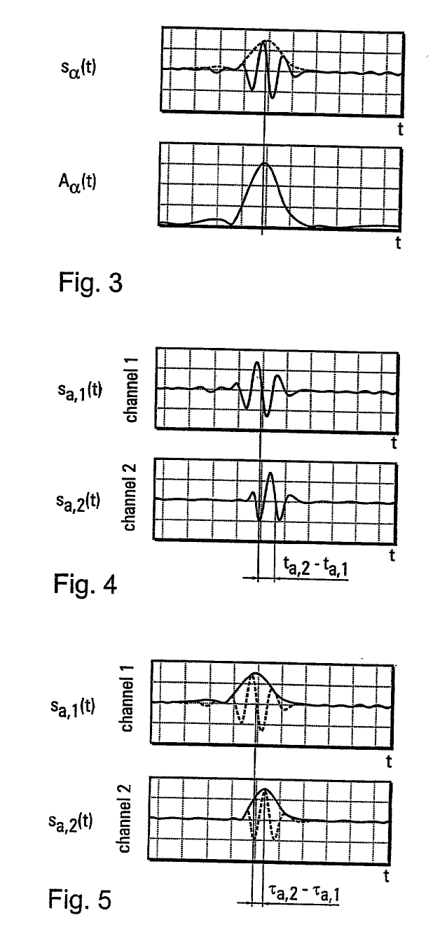

FIG. 3 shows a wavelet component Sₐ(t) and the corresponding envelope Aₐ(t) thereof. The envelope is shown in the form of a broken line once again in the upper diagram besides the wavelet component sₐ(t). It closely follows the curve configuration of the wavelet components and envelopes it upwardly. The envelope for a wavelet component is ascertained in the speed analysis unit 13. For that purpose, firstly the function ŝₐ(t) which is Hilbert-conjugated in respect of the wavelet components sₐ(t) is calculated on the basis of

$${\hat{s}}_{a} ( t ) = {π}^{- 1} {\int }_{- ∞}^{∞} \frac{{s}_{a} ( t )}{τ - t} ⅆ τ .$$

The envelope then derives from

$${A}_{a} ( t ) = \sqrt{{s}_{a}^{2}} ( t ) + {\hat{s}}_{a}^{2} ( t )$$

FIG. 4 shows two mutually corresponding wavelet components of the excitation wave signal recorded at the first and second electrodes. The diagram identified by channel 1 shows the wavelet component of the signal recorded by the first electrode and the diagram identified by channel 2 in turn shows the wavelet component of the signal recorded by the second electrode. For the purposes of ascertaining the phase speed of the wavelet components the shift in respect of time between the characteristic zero-passages of the illustrated wavelet components is ascertained. That is again effected in the speed analysis unit 13. The time difference between the zero-passages is identified by t_{a,2}-t_{a,1}. The speed analysis unit 13 ascertains the phase speed of the illustrated wavelet components from the spacing between the detection points r₁ and r₂, of the electrodes, and the above-identified time shift.

FIG. 5 shows two diagrams which are arranged one above the other and which are denoted by channel 1 and channel 2 and which respectively show a wavelet component (broken line) with the corresponding envelope (solid line). Channel 1 shows the wavelet component and the associated envelope for the signal recorded by the first electrode while channel 2 shows the corresponding curves for , the signal recorded by the second electrode. The maximum of the envelope of the first channel (channel 1) is identified by τ_{a,1} and the maximum of the envelope of the second channel (channel 2) is identified by τ_{a,2}. The time shift between the maxima of the envelopes is calculated from a Calculation of the time shifts of the maxima of the envelopes of the corresponding wavelet components of the first and second electrodes is implemented by the speed analysis unit. The so-called group speed of the wavelet components of the excitation wave results from the quotient between the spacing of the measurement points r₁ and r₂ and the above-described time shift of the maxima of the envelopes.

What is claimed is:

1. An apparatus for characterizing a condition of a myocardium, said apparatus comprising:
an excitation wave detector which detects an electrical excitation wave which is propagated through the myocardium at a first (r₁) and a second point (r₂) of the myocardium as a first signal (S₁(t)) and a second signal (S₂(t)); and
an analysis means, connected to the excitation wave detector, to analyze the first signal (S₁(t)) and the second signal (S₂(t)),
wherein the analysis means detects a difference between a signal shape of the first signal (S₁(t)) and the second signal (S₂(t)).

2. The apparatus of claim 1, wherein
the analysis means comprises at least one parameter unit to characterize the signal shape of the first signal (S₁(t)) and the second signal (S₂(t) on the basis of at least one parameter.

3. The apparatus of claim 2, wherein
the analysis means represents the first signal (S₁(t)) and the second signal (S₂(t)) by a superimposition of a set of functions {f(wt)} with wεR, wherein
$${S}_{1} ( t ) = {\int }_{- ∞}^{∞} {C}_{1} ( w ) f ( wt ) ⅆ w and {S}_{2} ( t ) = {\int }_{- ∞}^{∞} {C}_{2} ( w ) f ( wt ) ⅆ w .$$

4. The apparatus of claim 3, wherein
the analysis means comprises a Fourier analysis unit that effects a Fourier analysis, wherein f(wt)=exp(iwt) is to be used for the functions, and
$${C}_{1} ( w ) = {\int }_{- ∞}^{∞} {S}_{1} ( t ) exp ( - iwt ) ⅆ t and {C}_{2} ( w ) = {\int }_{- ∞}^{∞} {S}_{2} ( t ) exp ( - iwt ) ⅆ t .$$

5. The apparatus of claim 4, wherein
the analysis means further comprises a speed analysis unit which is connected to the Fourier analysis unit and ascertains a phase speed (vₚ(w)) of a Fourier component of the Fourier analysis.

6. The apparatus of claim 5, wherein
the analysis means comprises an attenuation analysis unit which is connected to the Fourier analysis unit and ascertains attenuation δ(w) of a Fourier component of the Fourier analysis between the points r₁ and r₂.

7. The apparatus of claim 1, wherein
the analysis means comprises a wavelet analysis unit which is adapted for the signals S₁(t) and S₂(t) to calculate the wavelet components S_{a,1}(t) and S_{a,2}(t) which are given
$${s}_{a , 1} ( t ) = {\int }_{- ∞}^{∞} {a}^{- 1 / 2} {C}_{1} ( a , b ) Ψ ( \frac{t - b}{a} ) ⅆ b$$
and
$${s}_{a , 2} ( t ) = {\int }_{- ∞}^{∞} {a}^{- 1 / 2} {C}_{2} ( a , b ) Ψ ( \frac{t - b}{a} ) ⅆ b ,$$
ψ((t−b)/a) are in that respect wavelets and
$${C}_{1} ( a , b ) = {\int }_{- ∞}^{∞} {a}^{- 1 / 2} {S}_{1} ( t ) Ψ ( ( t - b ) / a ) ⅆ t$$
$$and$$
$${C}_{2} ( a , b ) = {\int }_{- ∞}^{∞} {a}^{- 1 / 2} {S}_{2} ( t ) Ψ ( ( t - b ) / a ) ⅆ t$$
and S₂(t).

8. The apparatus of claim 7, wherein
the attenuation analysis unit is connected to the wavelet analysis unit and ascertains attenuation
$$δ ( a ) = {\int }_{- ∞}^{∞} {s}_{a , 2}^{2} ( t ) ⅆ t / {\int }_{- ∞}^{∞} {s}_{a , 1}^{2} ( t ) ⅆ t$$
of the wavelet component sₐ(t) between the points r₁ and r₂.

9. The apparatus of claim 8, wherein
the speed analysis unit is connected to the wavelet analysis unit and ascertains a phase speed vₚ(a) of the wavelet component sₐ(t) by means of vₚ(a)=|r₂−r₁|/(t_{a,2}−t_{a,1}), wherein s_{a,1}(t_{a,1})=0 and s_{a,2}(t_{a,2})=0.

10. The apparatus of claim 9, wherein
the speed analysis unit is connected to the wavelet analysis unit and ascertains a group speed v_{g}(a) of the wavelet component Sₐ(t) by means of v_{g}(a)=|r₂−r₁|/(τ_{a,2}−τ_{a,1}) with max(A_{a,1}(t))=A_{a,1}(τ_{a,1}) of A_{a,1}(t) and max(A_{a,2}(t))=A_{a,2}(τ_{a,2}) of A_{a,2}(t), wherein A_{a,1}(t) and A_{a,2}(t) respectively represent the envelopes
$${A}_{a , 1} = {[ {s}_{a , 1}^{2} ( t ) + {\hat{S}}_{a , 1}^{2} ( t ) ]}^{1 / 2} and {A}_{a , 2} = {[ {s}_{a , 2}^{2} ( t ) + {\hat{S}}_{a , 2}^{2} ( t ) ]}^{1 / 2}$$
of the wavelet components and
$${\hat{s}}_{a , 1} ( t ) = - {π}^{- 1} {\int }_{- ∞}^{∞} \frac{{s}_{a , 1} ( t )}{τ - t} ⅆ τ and {\hat{s}}_{a , 2} ( t ) = - {π}^{- 1} {\int }_{- ∞}^{∞} \frac{{s}_{a , 2} ( t )}{τ - t} ⅆ τ .$$

11. The apparatus of claim 10, wherein
the analysis means comprises a refractive index analysis unit which is connected to the speed analysis unit and ascertains a refractive index n(a) by means of n(a)=v_{g}(a)/vₚ(a).

12. The apparatus of claim 1, wherein
the excitation wave detector has a first and a second electrode for detecting the first signal (S₁(t)) and the second signal (S₂(t)).

13. The apparatus of claim 12, wherein
the first and second electrodes are adapted to be placed endocardially.

14. The apparatus of claim 1, further comprising
a signal store which is connected to the excitation wave detector and the analysis means and provides intermediate storage of the first and second signals.

15. The apparatus of claim 1, wherein
the analysis means represents the first signal (S₁(t)) and the second signal (S₂(t)) separately for each cardiac cycle by the superimposition of the set of functions {f(wt)}.

16. A method of operating an apparatus for characterizing a condition of a myocardium, comprising the steps of:
detecting an electrical excitation wave at a first point r₁ and a second point r₂ of the myocardium as a first signal (S₁(t)) and a second signal (S₂(t)), and
calculating a difference between a signal shape of the first signal (S₁(t)) and the second signal (S₂(t)).

17. The method of claim 16, further comprising the step of:
representing the first signal (S₁(t)) and the second signal (S₂(t)) by a superimposition of a set of functions {f(wt)} with wεR, wherein
$${S}_{1} ( t ) = {\int }_{- ∞}^{∞} {C}_{1} ( w ) f ( wt ) ⅆ w and {S}_{2} ( t ) = {\int }_{- ∞}^{∞} {C}_{2} ( w ) f ( wt ) ⅆ w .$$

18. The method of claim 17, further comprising the step of:
implementing a Fourier analysis by using exp(iwt) for the functions f(wt), wherein
$${C}_{1} ( w ) = {\int }_{- ∞}^{∞} {S}_{1} ( t ) exp ( - iwt ) ⅆ t and {C}_{2} ( w ) = {\int }_{- ∞}^{∞} {S}_{2} ( t ) exp ( - iwt ) ⅆ t .$$

19. The method of claim 18, further comprising, the step of:
ascertaining a phase speed (vₚ(w)) of a Fourier component of the Fourier analysis.

20. The method of claim 19, further comprising the step of:
ascertaining attenuation δ(w) of a Fourier component of the Fourier analysis between the points r₁ and r₂.

21. The method of claim 16, further comprising the step of:
using wavelet components sₐ(t) for the signals S₁(t) and S₂(t), wherein
$${S}_{1} ( t ) = {\sum }_{k = - ∞}^{∞} {s}_{{a}_{k} , 1} ( t ) and {S}_{2} ( t ) = {\sum }_{k = - ∞}^{∞} {s}_{{a}_{k} , 2} ( t ) ( {a}_{k} = {2}^{k} ) ,$$
the wavelet components are given
$$by {s}_{{a}_{k} , 1} ( t ) = {\int }_{- ∞}^{∞} {a}_{k}^{- 1 / 2} {C}_{1} ( {a}_{k} , b ) Ψ ( \frac{t - b}{{a}_{k}} ) ⅆ b and$$
$${s}_{{a}_{k} , 2} ( t ) = {\int }_{- ∞}^{∞} {a}_{k}^{- 1 / 2} {C}_{2} ( {a}_{k} , b ) Ψ ( \frac{t - b}{{a}_{k}} ) ⅆ b ,$$
ψ((t−b)/a) are wavelets and C₁(a,b) and C₂(a,b) represent the respectively corresponding wavelet transforms
$${C}_{1} ( a , b ) = {\int }_{- ∞}^{∞} {a}^{- 1 / 2} {S}_{1} ( t ) Ψ ( ( t - b ) / a ) ⅆ t$$
and
$${C}_{2} ( a , b ) = {\int }_{- ∞}^{∞} {a}^{- 1 / 2} {S}_{2} ( t ) Ψ ( ( t - b ) / a ) ⅆ t of {S}_{1} ( t ) and {S}_{2} ( t ) .$$

22. The method of claim 21, further comprising the step of
ascertaining attenuation δ(a) of the wavelet component sₐ(t) between the points r₁ and r₂ by means of
$$δ ( a ) = {\int }_{- ∞}^{∞} {s}_{a , 2}^{2} ( t ) ⅆ t / {\int }_{- ∞}^{∞} {s}_{a , 1}^{2} ( t ) ⅆ t .$$

23. The method of claim 22, further comprising the step of:
ascertaining a phase speed vₚ(a) of the wavelet component sₐ(t) between the points r₁ and r₂ by means of vₚ(a)=|r₂−r₁|/(t_{a,2}−t_{a,1}), with s_{a,1}(t_{a,1})=0 and s_{a,2}(t_{a,2})=0.

24. The method of claim 23, further comprising the step of:
ascertaining a group speed v_{g}(a) of the wavelet component sₐ(t) by means of v_{g}(a)=|r₂−r₁|/(τ_{a,2}−τ_{a,1}) with max(A_{a,1}(t))=A_{a,1}(τ_{a,1}) of A_{a,1}(t) and max(A_{a,2}(t))=A_{a,2}(τ_{a,2}) of A_{a,2}(t), wherein A_{a,1}(t) and A_{a,2}(t) respectively represent envelopes
$${A}_{a , 1} = {[ {s}_{a , 1}^{2} ( t ) + {\hat{s}}_{a , 1}^{2} ( t ) ]}^{1 / 2} and {A}_{a , 2} = {[ {s}_{a , 2}^{2} ( t ) + {\hat{s}}_{a , 2}^{2} ( t ) ]}^{1 / 2} with$$
$${\hat{s}}_{a , 1} ( t ) = - {π}^{- 1} {\int }_{- ∞}^{∞} \frac{{s}_{a , 1} ( t )}{τ - t} ⅆ τ and {\hat{s}}_{a , 2} ( t ) = - {π}^{- 1} {\int }_{- ∞}^{∞} \frac{{s}_{a , 2} ( t )}{τ - t} ⅆ τ .$$

25. The method of claim 24, further comprising the step of:
calculating a refractive index n(a) by means of n(a)=v_{g}(a)/vₚ(a).

26. The method of claim 16, further comprising the step of:
representing the first signal (S₁(t)) and the second signal (S₂(t)) separately for each cardiac cycle by the superimposition of the set of functions.

27. The apparatus of claim 2, wherein
the analysis means represents the first signal (S₁(t)) and the second signal (S₂(t)) by a superimposition of a set of functions {f(wt)} with wεR, wherein
$${S}_{1} ( t ) = {\int }_{- ∞}^{∞} {C}_{1} ( w ) f ( wt ) ⅆ w and {S}_{2} ( t ) = {\int }_{- ∞}^{∞} {C}_{2} ( w ) f ( wt ) ⅆ w .$$

28. The apparatus of claim 1, wherein
the analysis means comprises a Fourier analysis unit that effects a Fourier analysis, wherein f(wt)=exp(iwt) is to be used for the functions, and
$${C}_{1} ( w ) = {\int }_{- ∞}^{∞} {S}_{1} ( t ) exp ( - iwt ) ⅆ t and {C}_{2} ( w ) = {\int }_{- ∞}^{∞} {S}_{2} ( t ) exp ( - iwt ) ⅆ t .$$

29. The apparatus of claim 27, wherein
the analysis means comprises a Fourier analysis unit that effects a Fourier analysis, wherein f(wt)=exp(iwt) is to be used for the functions, and
$${C}_{1} ( w ) = {\int }_{- ∞}^{∞} {S}_{1} ( t ) exp ( - iwt ) ⅆ t and {C}_{2} ( w ) = {\int }_{- ∞}^{∞} {S}_{2} ( t ) exp ( - iwt ) ⅆ t .$$

30. The apparatus of claim 28, wherein
the analysis means further comprises a speed analysis unit which is connected to the Fourier analysis unit and ascertains a phase speed (vₚ(w)) of a Fourier component of the Fourier analysis.

31. The apparatus of claim 29, wherein
the analysis means further comprises a speed analysis unit which is connected to the Fourier analysis unit and ascertains a phase speed (vₚ(w)) of a Fourier component of the Fourier analysis.

32. The apparatus of claim 4, wherein
the analysis means comprises an attenuation analysis unit which is connected to the Fourier analysis unit and ascertains attenuation δ(w) of a Fourier component of the Fourier analysis between the points r₁ and r₂.

33. The apparatus of claim 30, wherein
the analysis means comprises an attenuation analysis unit which is connected to the Fourier analysis unit and ascertains attenuation δ(w) of a Fourier component of the Fourier analysis between the points r₁ and r₂.

34. The apparatus of claim 31, wherein
the analysis means comprises an attenuation analysis unit which is connected to the Fourier analysis unit and ascertains attenuation δ(w) of a Fourier component of the Fourier analysis between the points r₁ and r₂.

35. The apparatus of claim 7, wherein
the speed analysis unit is connected to the wavelet analysis unit and ascertains a phase speed vₚ(a) of the wavelet component sₐ(t) by means of vₚ(a)|r₂−r₁|/(t_{a,2}−t_{a,1}), wherein s_{a,1}(t_{a,1})=0 and s_{a,2}(t_{a,2})=0.

36. The apparatus of claim 35, wherein
the speed analysis unit is connected to the wavelet analysis unit and ascertains a group speed v_{g}(a) of the wavelet component sₐ(t) by means of v_{g}(a)=|r₂−r₁|/(τ_{a,2}−τ_{a,1}) with max(A_{a,1}(t))=A_{a,1}(τ_{a,1}) of A_{a,1}(t) and max(A_{a,2}(t))=A_{a,2}(τ_{a,2}) of A_{a,2}(t), wherein A_{a,1}(t) and A_{a,2}(t) respectively represent the envelopes
$${A}_{a , 1} = {[ {s}_{a , 1}^{2} ( t ) + {\hat{s}}_{a , 1}^{2} ( t ) ]}^{1 / 2} and {A}_{a , 2} = {[ {s}_{a , 2}^{2} ( t ) + {\hat{s}}_{a , 2}^{2} ( t ) ]}^{1 / 2}$$
of the wavelet components and
$${\hat{s}}_{a , 1} ( t ) = - {π}^{- 1} {\int }_{- ∞}^{∞} \frac{{s}_{a , 1} ( t )}{τ - t} ⅆ τ and {\hat{s}}_{a , 2} ( t ) = - {π}^{- 1} {\int }_{- ∞}^{∞} \frac{{s}_{a , 2} ( t )}{τ - t} ⅆ τ .$$

37. The apparatus of claim 36, wherein
the analysis means comprises a refractive index analysis unit which is connected to the speed analysis unit and ascertains a refractive index n(a) by means of n(a)=v_{g}(a)/vₚ(a).

38. The method of claim 16, further comprising the step of:
implementing a Fourier analysis by using exp(iwt) for the functions f(wt), wherein
$${C}_{1} ( w ) = {\int }_{- ∞}^{∞} {S}_{1} ( t ) exp ( - iwt ) ⅆ t and {C}_{2} ( w ) = {\int }_{- ∞}^{∞} {S}_{2} ( t ) exp ( - iwt ) ⅆ t .$$

39. The method of claim 38, further comprising the step of:
ascertaining a phase speed (vₚ(w)) of a Fourier component of the Fourier analysis.

40. The method of claim 39, further comprising the step of:
ascertaining attenuation δ(w) of a Fourier component of the Fourier analysis between the points r₁ and r₂.

41. The method of claim 18, further comprising the step of:
ascertaining attenuation δ(w) of a Fourier component of the Fourier analysis between the points r₁ and r₂.

42. The method of claim 21, further comprising the step of:
ascertaining a phase speed vₚ(a) of the wavelet component sₐ(t) between the points r₁ and r₂ by means of vₚ(a)=|r₂−r₁|/(t_{a,2}−t_{a,1}), with s_{a,1}(t_{a,1})=0 and s_{a,2}(t_{a,2})=0.

43. The method of claim 21, further comprising the step of:
ascertaining a group speed v_{g}(a) of the wavelet component sₐ(t) by means of v_{g}(a)=|r₂−r₁|(τ_{a,2}−τ_{a,1}) with max(A_{a,1}(t))=A_{a,1}(τ_{a,1}) of A_{a,1}(t) and max(A_{a,2}(t))=A_{a,2}(τ_{a,2}) of A_{a,2}(t), wherein A_{a,1}(t) and A_{a,2}(t) respectively represent envelopes
$${A}_{a , 1} = {[ {s}_{a , 1}^{2} ( t ) + {\hat{s}}_{a , 1}^{2} ( t ) ]}^{1 / 2} and {A}_{a , 2} = {[ {s}_{a , 2}^{2} ( t ) + {\hat{s}}_{a , 2}^{2} ( t ) ]}^{1 / 2} with$$
$${\hat{s}}_{a , 1} ( t ) = - {π}^{- 1} {\int }_{- ∞}^{∞} \frac{{s}_{a , 1} ( t )}{τ - t} ⅆ τ and {\hat{s}}_{a , 2} ( t ) = - {π}^{- 1} {\int }_{- ∞}^{∞} \frac{{s}_{a , 2} ( t )}{τ - t} ⅆ τ .$$

44. The apparatus of claim 12, wherein
the first and second electrodes are adapted to be placed epicardially.
